(19) United States
(12) Patent Application Publication
(10) Pub. No.: US 2017/0055950 A1
(43) Pub. Date: Mar. 2, 2017

(54) ULTRASONIC DEVICE, ULTRASONIC MODULE, AND ULTRASONIC MEASUREMENT APPARATUS

(71) Applicant: Seiko Epson Corporation, Tokyo (JP)

(72) Inventor: Hiroshi MATSUDA, Gifu (JP)

(21) Appl. No.: 15/244,269

(22) Filed: Aug. 23, 2016

(30) Foreign Application Priority Data
Aug. 31, 2015 (JP) 2015-171174

(51) Int. Cl. A61B 8/00; A61B 8/14

(52) U.S. Cl. CPC A61B 8/4488; A61B 8/145; A61B 8/4427; A61B 8/4494; A61B 8/54

(57) ABSTRACT

An ultrasonic device includes a plurality of ultrasonic element groups each including at least one transmitting element adapted to transmit an ultrasonic wave and at least one receiving element adapted to receive an ultrasonic wave, and arranged along an X direction, and in each of the ultrasonic element groups, a centroid position of the receiving area, in which the receiving element included in the ultrasonic element group is disposed, overlaps a transmitting area, in which the transmitting element included in the ultrasonic element group is disposed, in a projection view along a Y direction.

### BACKGROUND

1. Technical Field

The present invention relates to an ultrasonic device, an ultrasonic module, and an ultrasonic measurement apparatus.

2. Related Art

In the past, there has been known an ultrasonic diagnostic device (ultrasonic measurement device) for transmitting and receiving an ultrasonic wave using an ultrasonic probe to thereby form an ultrasonic image (see, e.g., JP-A-2011-160856 (Document 1)).

In the device described in Document 1, the ultrasonic probe has a transmitting array and a receiving array. Among these arrays, the transmitting array is configured as a one-dimensional array having a plurality of fundamental resonators, which correspond to an ultrasonic wave as a fundamental wave, arranged in one direction (a scanning direction) in accordance with an arrangement condition corresponding to the fundamental wave. Further, the receiving array is configured as a one-dimensional array having a plurality of harmonic resonators, which correspond to an ultrasonic wave as a high-order harmonic wave with respect to the fundamental wave, arranged in the one direction described above in accordance with a predetermined arrangement condition corresponding to the order of a high-order harmonic wave. The transmitting array and the receiving array are arranged in parallel and close to each other.

However, in the ultrasonic probe described in Document 1, a receiving aperture constituted by the receiving array is separated in a slicing direction perpendicular to a scanning direction from a transmitting aperture constituted by the transmitting array. In other words, the central positions of the transmitting aperture and the receiving aperture in the slicing direction are different from each other, and in particular in the configuration of Document 1, the central position of the receiving aperture is located outside the transmitting aperture.

Therefore, it is necessary to converge the ultrasonic wave (a transmission wave) transmitted from the transmitting aperture so that the convergence region (a so-called hotspot) of the transmission wave is located on the receiving aperture side in the slicing direction from the position overlapping the central position of the transmitting aperture in the normal direction of the ultrasonic array. Therefore, since the acoustic intensity at the hotspot is lower compared to the case in which the central positions of the transmitting aperture and the receiving aperture coincide with each other in the slicing direction, and thus the resolution degrades, the accuracy of the ultrasonic measurement degrades.

Further, if the ultrasonic wave is transmitted in the direction tilted at a predetermined angle with the slicing direction of the ultrasonic array as described above, it is necessary to perform signal processing of the received signal and image processing taking the angle variation into consideration in the case of generating an internal tomographic image of an object based on the reception result of the ultrasonic wave received by the receiving array, and therefore, there is a problem that the process becomes complicated.

### SUMMARY

An advantages of some aspects of the invention is to provide an ultrasonic device, an ultrasonic module, and an ultrasonic measurement apparatus each capable of performing highly accurate ultrasonic measurements with a simple process.

An ultrasonic device according to an application example of the invention includes a plurality of element groups each including at least one transmitting element adapted to transmit an ultrasonic wave and at least one receiving element adapted to receive an ultrasonic wave, and arranged along a first direction, and in each of the element groups, a centroid position of a receiving area, in which the receiving element included in the element group is disposed, overlaps a transmitting area, in which the transmitting element included in the element group is disposed, in a projection view along the first direction.

In this application example, the element groups each have the transmitting element and the receiving element, and are configured so as to be able to transmit and receive the ultrasonic wave, and are arranged along the first direction. In these element groups, the centroid position of the receiving area overlaps the transmitting area in the projection view along the first direction.

Here, in the case in which the transmitting area and the receiving area are arranged so as to be shifted from each other in a second direction crossing the first direction when viewed in the first direction, since it is necessary to transmit the ultrasonic wave so as to be tiled toward the receiving area in the second direction, there is a possibility that it is not achievable to sufficiently converge the transmission wave, and thus, the resolution degrades.

Further, in the case of transmitting the ultrasonic wave so as to be tilted toward the receiving area in the second direction, and receiving the reflected ultrasonic wave, which has been reflected by the measurement object, in the receiving area, the entering angle (the incident angle) to the receiving area of the reflected ultrasonic wave to be received by the receiving area varies. Therefore, it is necessary to consider the influence of the variation of the incident angle such as a variation in the delay time of the reflected wave in accordance with the incident angle when performing the signal processing based on the reception signal, and there is a possibility that the signal processing and the image processing based on the signal processing become complicated.

In contrast, in this application example, since the centroid position of the receiving area overlaps the transmitting area when viewed in the first direction, the acoustic intensity at the hotspot can be enhanced, and thus, the degradation of the resolution described above can be suppressed. Further, if the ultrasonic wave is transmitted from the transmitting area toward the roughly normal direction, the ultrasonic wave reflected by the object can be received in the receiving area. Therefore, a variety of types of signal processing can be performed without considering the influence of the variation of the incident angle, and the complication of the process can be suppressed, and thus, the ultrasonic image can be formed with a simple process.

Therefore, according to this application example, it is possible to provide the ultrasonic device capable of performing a highly accurate ultrasonic measurement with a simple process.

In the ultrasonic device according to the application example, it is preferable that the receiving area is located inside the transmitting area in the projection view along the first direction.

In the application example with this configuration, the receiving area is located inside the transmitting area in the projection view along the first direction. According to this configuration, the degradation of the resolution and the complication of the signal processing and the image processing due to the shift in the second direction between the transmitting area and the receiving area can be suppressed as described above.

In the ultrasonic device according to the application example, it is preferable that a centroid position of the receiving area overlaps a centroid position of the transmitting area in the projection view along the first direction.

In the application example with this configuration, the centroid position of the receiving area coincides with the centroid position of the transmitting area in the projection view along the first direction. According to this configuration, the degradation of the resolution and the complication of the signal processing and the image processing due to the shift in the second direction between the centroid positions of the transmitting area and the receiving area can more surely be suppressed as described above.

In the ultrasonic device according to the application example, it is preferable that the centroid position of the receiving area coincides with the centroid position of the transmitting area.

In the application example with this configuration, the centroid positions of the transmitting area and the receiving area further coincide with each other. Therefore, the degradation of the resolution can more surely be suppressed in both of the first direction and the second direction.

In the ultrasonic device according to the application example, it is preferable that the transmitting elements included in the element group are disposed at positions axisymmetrical about an imaginary line passing through the centroid position of the transmitting area and parallel to the first direction, and the receiving elements included in the element group are disposed at positions axisymmetrical about the imaginary line.

In the application example with this configuration, each of the transmitting elements and the receiving elements are disposed at positions axisymmetrical about an imaginary line passing through the centroid position of the transmitting area (the receiving area) and parallel to the first direction. According to this configuration, since the symmetrical property in the second direction of the transmitting area and the receiving area can be improved, the resolution in the second direction can be improved, and thus, the reception accuracy can be improved.

In the ultrasonic device according to the application example, it is preferable that the transmitting elements included in the element group are disposed at positions point-symmetrical about the centroid position of the transmitting area, and the receiving elements included in the element group are disposed at positions point-symmetrical about the centroid position of the transmitting area.

In the application example with this configuration, the transmitting elements are disposed at the positions point-symmetrical about the centroid position of the transmitting area, and the receiving elements are disposed at the positions point-symmetrical about the centroid position of the receiving area. According to this configuration, the planar symmetry of the transmitting area and the receiving area can be improved. Therefore, it is possible to converge the ultrasonic wave transmitted from the transmitting elements to thereby improve the resolution in the case of receiving the reflected wave, which has been reflected by the object, with the receiving elements, and thus, the reception accuracy can be improved.

In the ultrasonic device according to the application example, it is preferable that the element group has an array structure in which ultrasonic elements including the transmitting element and the receiving element are arranged in a two-dimensional array along the first direction and a second direction crossing the first direction.

In the application example with this configuration, since the ultrasonic elements are arranged along the first direction and the second direction, the planar symmetry in the arrangement of the ultrasonic elements can further be improved. Therefore, it is possible to transmit the ultrasonic wave three-dimensionally more uniform from the transmitting elements, and thus, the accuracy of the ultrasonic measurement can be improved.

In the ultrasonic device according to the application example, it is preferable that the element groups each have a plurality of receiving elements, the plurality of receiving elements includes at least one first receiving element adapted to receive a high-order harmonic wave taking the ultrasonic wave transmitted from the transmitting element as a fundamental wave, and at least one second receiving element adapted to receive a high-order harmonic wave of a different order from an order of the high-order harmonic wave received by the first receiving element, the first receiving elements are disposed at positions point-symmetrical about the centroid position of the receiving area, and the second receiving elements are disposed at positions point-symmetrical about the centroid position of the receiving area.

In the application example with this configuration, as the receiving elements, the first receiving elements and the second receiving elements for receiving the high-order harmonic waves of the respective orders different from each other are provided. Further, the first receiving elements and the second receiving elements are each disposed at the positions point-symmetrical about the centroid position of the receiving area. Thus, each of the high-order harmonic waves different in order can be received with high accuracy.

An ultrasonic module according to an application example of the invention includes an ultrasonic device including a plurality of element groups each including at least one transmitting element adapted to transmit an ultrasonic wave and at least one receiving element adapted to receive an ultrasonic wave, and arranged along a first direction, and a circuit board on which the ultrasonic device is disposed, and in each of the element groups, a centroid position of a receiving area, in which the receiving element included in the element group is disposed, overlaps a transmitting area, in which the transmitting element included in the element group is disposed, in a projection view along the first direction.

In this application example, similarly to the application examples described above, the element groups each have the transmitting element and the receiving element, and are configured so as to be able to transmit and receive the ultrasonic wave, and are arranged along the first direction. In these element groups, the centroid position of the receiving area overlaps the transmitting area in the projection view along the first direction.

Thus, the acoustic intensity at the hotspot can be enhanced, and thus, the degradation of the resolution described above can be suppressed. Further, if the ultrasonic wave is transmitted from the transmitting area toward the roughly normal direction, the ultrasonic wave reflected by the object can be received in the receiving area. Therefore, a variety of types of signal processing can be performed without considering the influence of the variation of the incident angle of the reflected ultrasonic wave to the receiving area in accordance with the reflection position of the ultrasonic wave, and the complication of the process can be suppressed, and thus, the ultrasonic image can be formed with a simple process.

Therefore, according to this application example, it is possible to provide the ultrasonic module capable of performing a highly accurate ultrasonic measurement with a simple process.

An ultrasonic measurement apparatus according to an application example of the invention includes an ultrasonic device including a plurality of element groups each including at least one transmitting element adapted to transmit an ultrasonic wave and at least one receiving element adapted to receive an ultrasonic wave, and arranged along a first direction, and a control section adapted to control the ultrasonic device, and in each of the element groups, a centroid position of a receiving area, in which the receiving element included in the element group is disposed, overlaps a transmitting area, in which the transmitting element included in the element group is disposed, in a projection view along the first direction.

In this application example, similarly to the application examples described above, the element groups each have the transmitting element and the receiving element, and are configured so as to be able to transmit and receive the ultrasonic wave, and are arranged along the first direction. In these element groups, the centroid position of the receiving area overlaps the transmitting area in the projection view along the first direction.

Thus, the acoustic intensity at the hotspot can be enhanced, and thus, the degradation of the resolution described above can be suppressed. Further, if the ultrasonic wave is transmitted from the transmitting area toward the roughly normal direction, the ultrasonic wave reflected by the object can be received in the receiving area. Therefore, a variety of types of signal processing can be performed without considering the influence of the variation of the incident angle of the reflected ultrasonic wave to the receiving area in accordance with the reflection position of the ultrasonic wave, and the complication of the process can be suppressed, and thus, the ultrasonic image can be formed with a simple process.

Therefore, according to this application example, it is possible to provide the ultrasonic measurement apparatus capable of performing a highly accurate ultrasonic measurement with a simple process.

### BRIEF DESCRIPTION OF THE DRAWINGS

The invention will be described with reference to the accompanying drawings, wherein like numbers reference like elements.

FIG. 1 is a perspective view showing a general configuration of an ultrasonic measurement apparatus according to a first embodiment of the invention.

FIG. 2 is a block diagram showing a general configuration of an ultrasonic measurement apparatus according to the embodiment.

FIG. 3 is a cross-sectional view showing a schematic configuration of an ultrasonic probe according to the embodiment.

FIG. 4 is a plan view showing a schematic configuration of an ultrasonic device according to the embodiment.

FIGS. 5A through 5C are cross-sectional views showing the schematic configuration of the ultrasonic device according to the embodiment.

FIG. 6 is a plan view showing a schematic configuration of an ultrasonic element group of the embodiment.

FIG. 7 is a diagram showing an operational example of the ultrasonic element group of the embodiment.

FIG. 8 is a diagram showing an operational example of an ultrasonic device according to a comparative example.

FIG. 9 is a plan view showing a schematic configuration of an ultrasonic element group of a modified example of the first embodiment.

FIG. 10 is a plan view showing a schematic configuration of an ultrasonic element group of a second embodiment of the invention.

FIG. 11 is a plan view showing a schematic configuration of an ultrasonic element group of a third embodiment of the invention.

FIG. 12 is a plan view showing a schematic configuration of an ultrasonic element group of a fourth embodiment of the invention.

FIG. 13 is a plan view showing a schematic configuration of an ultrasonic element group related to a modified example of the invention.

### DESCRIPTION OF EXEMPLARY EMBODIMENTS

### First Embodiment

An ultrasonic measurement apparatus as an electronic apparatus of a first embodiment according to the invention will hereinafter be described based on the accompanying drawings.

### Configuration of Ultrasonic Measurement Apparatus

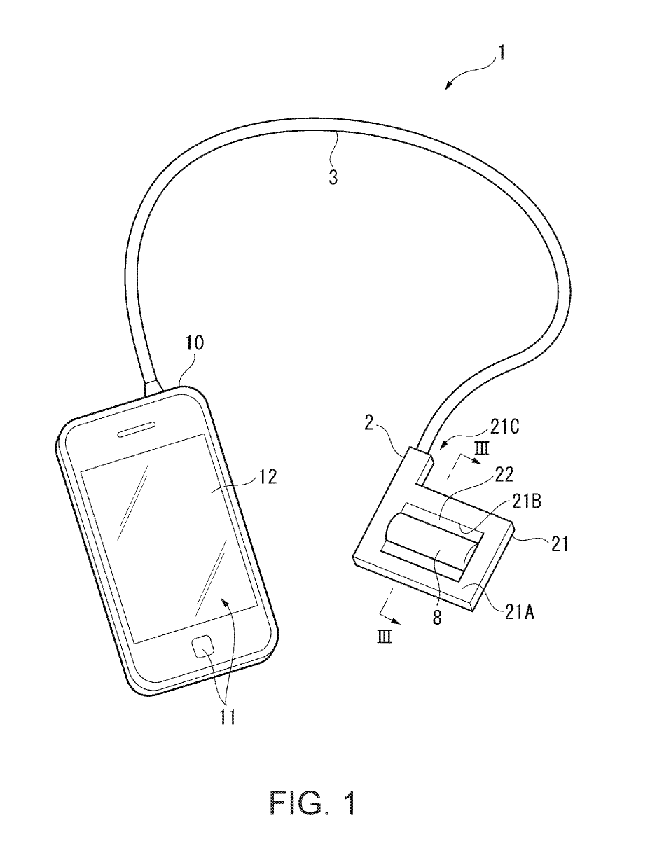

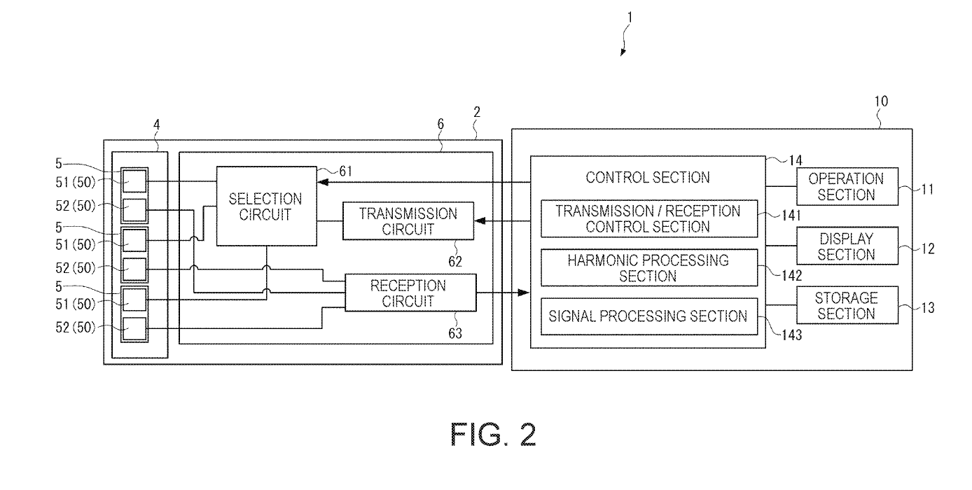

FIG. 1 is a perspective view showing a general configuration of the ultrasonic measurement apparatus 1 according to the present embodiment. FIG. 2 is a block diagram showing a general configuration of the ultrasonic measurement apparatus 1.

As shown in FIG. 1, the ultrasonic measurement apparatus 1 according to the present embodiment is provided with an ultrasonic probe 2, and a control device 10 electrically connected to the ultrasonic probe 2 via a cable 3.

The ultrasonic measurement apparatus 1 transmits an ultrasonic wave from the ultrasonic probe 2 to the inside of a living body (e.g., a human body) with the ultrasonic probe 2 having contact with a surface of the living body. Further, the ultrasonic measurement apparatus 1 receives a high-order harmonic wave with respect to the fundamental wave out of the ultrasonic wave reflected by a part in the living body using the ultrasonic probe 2, and then obtains an internal tomographic image in the living body, for example, and measures the state (e.g., blood flow) of the part in the living body based on the received signal.

### Configuration of Ultrasonic Probe

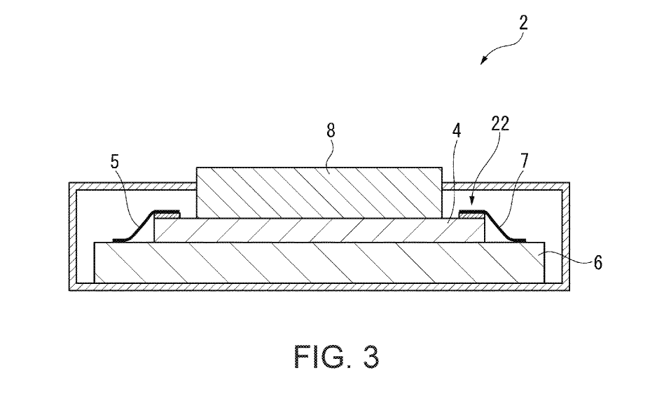

FIG. 3 is a cross-sectional view of the ultrasonic probe 2 cut along the line III-III shown in FIG. 1, and showing a general configuration of the ultrasonic probe 2.

The ultrasonic probe 2 is provided with a housing 21 and an ultrasonic sensor 22.

### Configuration of Housing

As shown in FIG. 1, the housing 21 is formed to have a box-like shape having a rectangular planar shape, and supports the ultrasonic sensor 22. One surface (a sensor surface 21A) perpendicular to the thickness direction of the housing 21 is provided with a sensor window 21B, and a part (an acoustic lens 8 described later) of the ultrasonic sensor 22 is exposed. Further, in apart (a side surface in the example shown in FIG. 1) of the housing 21, there is disposed a through hole 21C for the cable 3, and the cable 3 is inserted into the housing 21. Although not shown in the drawings, the cable 3 is connected to the ultrasonic sensor 22 (a circuit board 6 described later) in the inside of the housing 21. Further, the gap between the cable 3 and the through hole 21C is filled with, for example, a resin material to thereby ensure the waterproof property.

It should be noted that although in the present embodiment, there is shown a configuration example in which the ultrasonic probe 2 and the control device 10 are connected to each other using the cable 3, the configuration is not limited to this example, and it is also possible to, for example, connect the ultrasonic probe 2 and the control device 10 to each other with wireless communication, or dispose a variety of constituents of the control device 10 inside the ultrasonic probe 2.

### Configuration of Ultrasonic Sensor

The ultrasonic sensor 22 corresponds to the ultrasonic module according to the invention, and is provided with an ultrasonic device 4, the circuit board 6, a flexible board 7, and the acoustic lens 8 as shown in FIG. 3. Although described later, the circuit board 6 is provided with a driver circuit for controlling the ultrasonic device 4, and so on, and the ultrasonic device 4 is electrically connected to the circuit board 6 via the flexible board 7. On the ultrasonic wave transmission/reception side surface of the ultrasonic device 4, there is disposed the acoustic lens 8. The ultrasonic sensor 22 is housed in the housing 21 so that the acoustic lens 8 is exposed, transmits the ultrasonic wave from the exposed part to an object, and then receives a reflected wave from the object.

### Configuration of Acoustic Lens

The acoustic lens 8 efficiently propagates the ultrasonic wave, which has been emitted from the ultrasonic device 4, to the living body as the measurement object, and further propagates the ultrasonic wave, which has been reflected in the living body, to the ultrasonic device 4 with efficiency. The acoustic lens 8 is disposed along the surface with which the ultrasonic device 4 transmits and receives the ultrasonic wave. It should be noted that although not shown in the drawings, between the ultrasonic device 4 and the acoustic lens 8, there is disposed an acoustic matching layer. The acoustic lens 8 and the acoustic matching layer are set to have an acoustic impedance intermediate between the acoustic impedance of the ultrasonic elements 50 (transmitting element 51 and receiving element 52) of an element substrate 41 and the acoustic impedance of the living body.

### Configuration of Ultrasonic Device

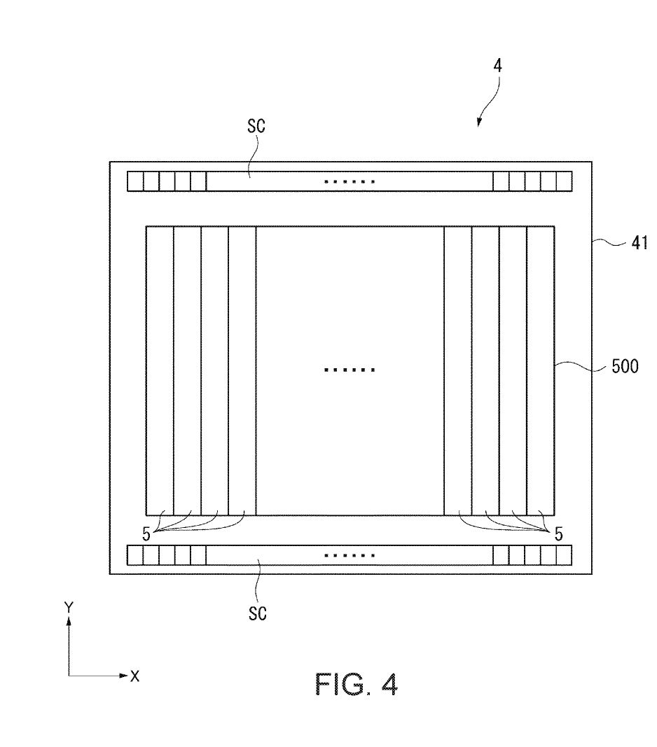

FIG. 4 is a plan view of the ultrasonic device 4 viewed from the acoustic lens 8 side, and showing a schematic configuration of the ultrasonic device 4.

In the following description, the scanning direction (a first direction) of the ultrasonic device 4 having a one-dimensional array structure as described later is defined as an X direction, and the slicing direction (a second direction) perpendicular to the scanning direction is defined as a Y direction.

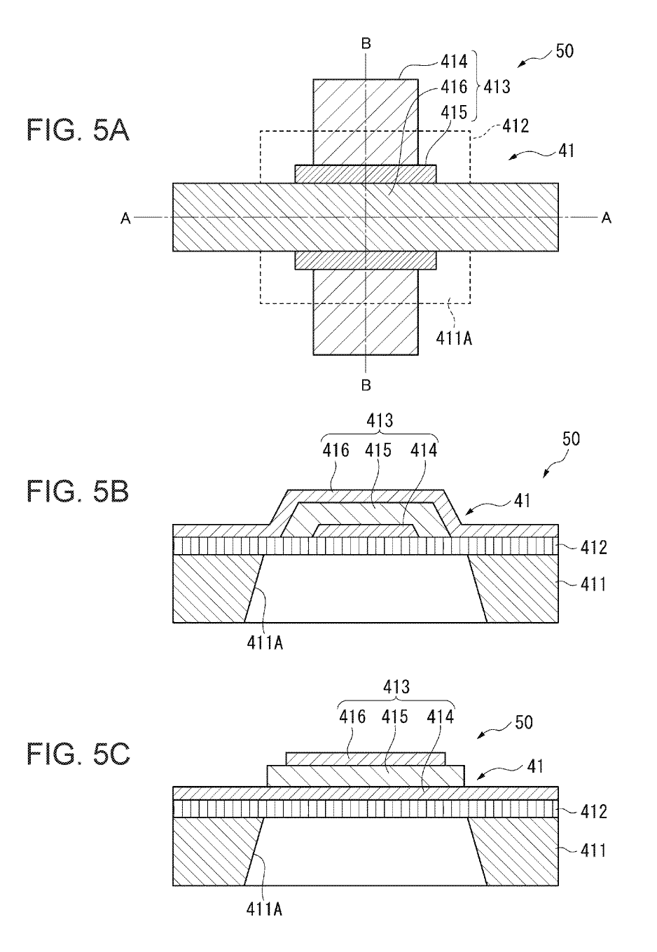

The ultrasonic device 4 is constituted by a plurality of ultrasonic element groups 5, each of which has a plurality of ultrasonic elements 50 (see FIGS. 5A through 5C), and which are arranged in the Y direction on the element substrate 41. The ultrasonic element groups 5 arranged in the slicing direction are each a transmission/reception channel for transmitting and receiving the ultrasonic wave using the ultrasonic elements 50. The ultrasonic element groups 5 as the transmission/reception channels are arranged in the Y direction to constitute an ultrasonic array 500 as the one-dimensional array.

It should be noted that the element substrate 41 is provided with wiring (not shown) for driving the plurality of ultrasonic elements 50, and signal terminals Sc, to which the wiring is connected, and which are connected to the circuit board 6 with the flexible board 7. It should be noted that in the example shown in the drawing, the signal terminals Sc are disposed in the end parts on the both sides in the Y direction of the element substrate 41 along the X direction.

### Configuration of Element Substrate

FIGS. 5A through 5C show an example of a configuration of the element substrate 41 and the ultrasonic element 50. FIG. 5A is a plan view of the ultrasonic element 50, FIG. 5B is a cross-sectional view showing a cross-sectional surface along the line B-B shown in FIG. 5A, and FIG. 5C is a cross-sectional view showing a cross-sectional surface along the line C-C shown in FIG. 5A.

As shown in FIGS. 5A through 5C, the element substrate 41 is provided with a substrate main body part 411, and a vibrating film 412 stacked on the substrate main body part 411.

The substrate main body part 411 is a semiconductor substrate made of, for example, Si. Inside the array region of the substrate main body part 411, there are disposed aperture parts 411A corresponding respectively to the ultrasonic elements. Further, the aperture parts 411A are closed by the vibrating film 412 disposed on the rear surface 41A side of the substrate main body part 411.

The vibrating film 412 is formed of, for example, SiO₂ or a laminated body of SiO₂ and ZrO₂, and is disposed so as to cover the entire area on the rear surface 41A side of the substrate main body part 411. The thickness dimension of the vibrating film 412 becomes sufficiently small one with respect to the substrate main body part 411. In the case of forming the substrate main body part 411 using Si and forming the vibrating film 412 using SiO₂, by performing an oxidation treatment on, for example, the surface on the rear surface 41A side of the substrate main body part 411, it becomes possible to easily form the vibrating film 412 having a desired thickness dimension. Further, in this case, by performing an etching treatment on the substrate main body part 411 using the vibrating film 412 made of SiO₂ as an etching stopper, it is possible to easily form the aperture parts 411A.

### Configuration of Ultrasonic Wave Transmitting Sections

As shown in FIGS. 5A through 5C, the ultrasonic elements 50 are each configured including the vibrating film 412 and a piezoelectric element 413.

The piezoelectric element 413 is a laminated body of a lower-part electrode 414, a piezoelectric film 415, and an upper-part electrode 416, and is disposed on the vibrating film 412 closing each of the aperture parts 411A as shown in FIGS. 5A through 5C.

The lower-part electrode 414 is connected to the signal terminal via a signal line not shown, and a drive voltage is applied to the lower-part electrode 414. It should be noted that the lower-part electrodes 414 of the transmitting elements 51 described later out of the ultrasonic elements 50 included in one transmission/reception channel are electrically connected to each other via the signal lines. Similarly, the lower-part electrodes 414 of the receiving elements 52 described later out of the ultrasonic elements 50 included in one transmission/reception channel are electrically connected to each other via the signal lines.

The piezoelectric film 415 is formed of a thin film made of lead zirconate titanate (PZT) or the like, and is configured so as to cover at least the lower-part electrode 414.

The upper-part electrode 416 is connected to a common terminal via the signal line not shown, and a common voltage is applied to the upper-part electrode 416. It should be noted that the plurality of upper-part electrodes 416 is electrically connected via the signal lines.

Here, the ultrasonic elements 50 include the transmitting elements 51 for transmitting the ultrasonic wave (fundamental wave) and the receiving elements 52 capable of receiving a high-order harmonic wave with respect to the fundamental wave as described later.

The transmitting elements 51 are each configured so as to be able to transmit the fundamental wave having a predetermined frequency at a desired efficiency. Specifically, in the transmitting element 51, by applying a rectangular-wave voltage having a predetermined frequency between the lower-part electrode 414 and the upper-part electrode 416, it is possible to vibrate the vibrating film 412 in an aperture region of each of the aperture parts 411A to transmit the ultrasonic wave. In the transmitting element 51, the shape (the aperture shape of the aperture part 411A) of the vibrating film 412 is set in accordance with the frequency of the fundamental wave, and the fundamental wave can be transmitted at a desired efficiency.

The receiving element 52 is configured so as to be able to receive the high-order harmonic wave having the predetermined order with respect to the fundamental wave at a desired efficiency, but is basically configured similarly to the transmitting element 51, and has the shape of the vibrating film 412 set in accordance with the frequency of the high-order harmonic wave with respect to the fundamental wave, and is capable of receiving the high-order harmonic wave at a desired efficiency.

### Configuration of Ultrasonic Element Group

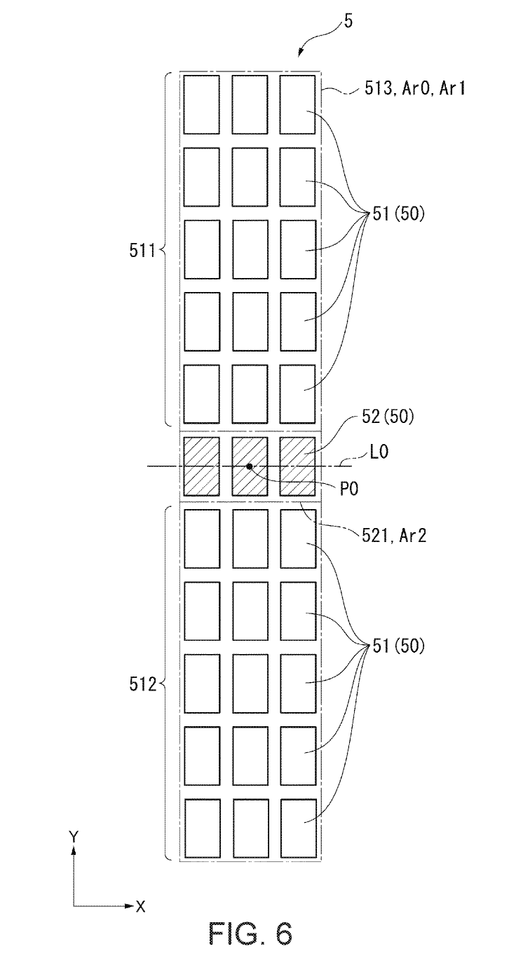

FIG. 6 is a plan view showing a schematic configuration of the ultrasonic wave element group 5 when viewing the element substrate 41 from the acoustic lens 8 side.

The ultrasonic element group 5 is constituted by the plurality of ultrasonic elements 50 including the transmitting elements 51 and the receiving elements 52 arranged in a two-dimensional array along the X direction and the Y direction. The ultrasonic element group 5 receives a high-order harmonic wave with respect to the fundamental wave having been transmitted from the transmitting elements 51 using the receiving elements 52. It should be noted that in the following description, the area surrounding the arrangement positions of the plurality of ultrasonic elements 50 constituting the ultrasonic element group 5 is defined as a transmitting/receiving area Ar0. The transmitting/receiving area Ar0 is the maximum area of rectangular areas surrounding the plurality of ultrasonic elements 50 constituting the ultrasonic element group 5 in a circumscribed manner in a planar view viewed from the thickness direction of the element substrate 41, for example. In other words, the transmitting/receiving area Ar0 is the minimum area of the rectangular areas surrounding all of the ultrasonic elements 50 constituting the ultrasonic element group 5. Further, the center of the transmitting/receiving area Ar0 is defined as a central position P0, and an imaginary line passing through the central position P0 and parallel to the X direction is defined as a center line L0.

The transmitting elements 51 are arranged in the X direction and the Y direction except on the center line L0. Among the plurality of transmitting elements 51, the transmitting elements 51 disposed on the +Y side of the center line L0 constitute a first transmitting aperture 511, and the transmitting elements 51 disposed on the −Y side of the center line L0 constitute a second transmitting aperture 512.

The first transmitting aperture 511 and the second transmitting aperture 512 constitute a composite transmitting aperture 513. A first area Ar1 in which the composite transmitting aperture 513 is set is a rectangular area (transmitting area) surrounding the first transmitting aperture 511 and the second transmitting aperture 512. In the present embodiment, the first area Ar1 coincides with the transmitting/receiving area Ar0. Similarly to the transmitting/receiving area described above, the transmitting area is the maximum area of the rectangular areas surrounding the plurality of transmitting elements 51 constituting the ultrasonic element group 5 in a circumscribed manner in the planar view. In other words, the transmitting area is the minimum area of the rectangular areas surrounding all of the transmitting elements 51.

Further, the transmitting elements 51 are arranged so that the first transmitting aperture 511 and the second transmitting aperture 512 become in an axisymmetrical relationship about the center line L0, and become point-symmetrical about the central position P0. Therefore, the centroid position of the area (the transmitting area) in which the transmitting elements 51 are arranged coincides with the central position P0. The transmitting elements 51 are disposed at positions, which are axisymmetrical about the center line L0 and at the same time point-symmetrical about the central position P0 as the centroid position.

The receiving elements 52 are arranged in the X direction along the center line L0 to constitute a receiving aperture 521. A second area Ar2 set as the receiving aperture 521 is a rectangular area (a receiving area) surrounding the receiving aperture 521, and is included in the transmitting/receiving area Ar0 in a planar view viewed from the normal direction of the element substrate 41. Similarly to the transmitting/receiving area described above, the receiving area is the maximum area of the rectangular areas surrounding the plurality of receiving elements 52 constituting the ultrasonic element group 5 in a circumscribed manner in the planar view. In other words, the receiving area is the minimum area of the rectangular areas surrounding all of the receiving elements 52.

In the receiving aperture 521, the receiving elements 52 are arranged so as to be axisymmetrical about the center line L0 and point-symmetrical about the central position P0. Therefore, the centroid position of the area (the receiving area) in which the receiving elements 52 are arranged coincides with the central position P0. The receiving elements 52 are disposed at positions, which are axisymmetrical about the center line L0 and at the same time point-symmetrical about the central position P0 as the centroid position. Further, the receiving aperture 521 (the receiving area) is located inside the composite transmitting aperture 513 (the transmitting area) viewed in the X direction.

### Transmission/Reception of Ultrasonic Wave by Ultrasonic Element Group

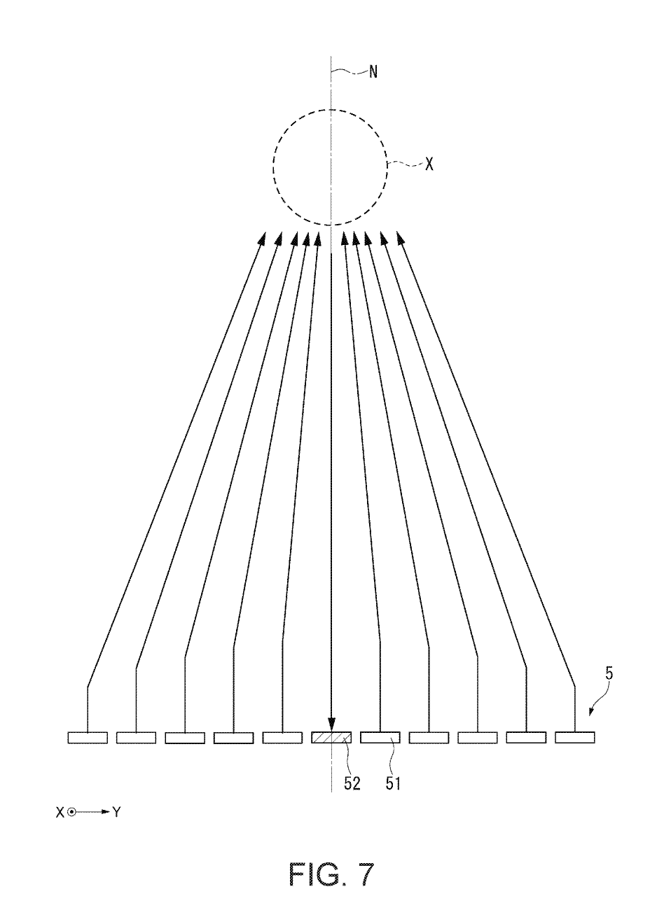

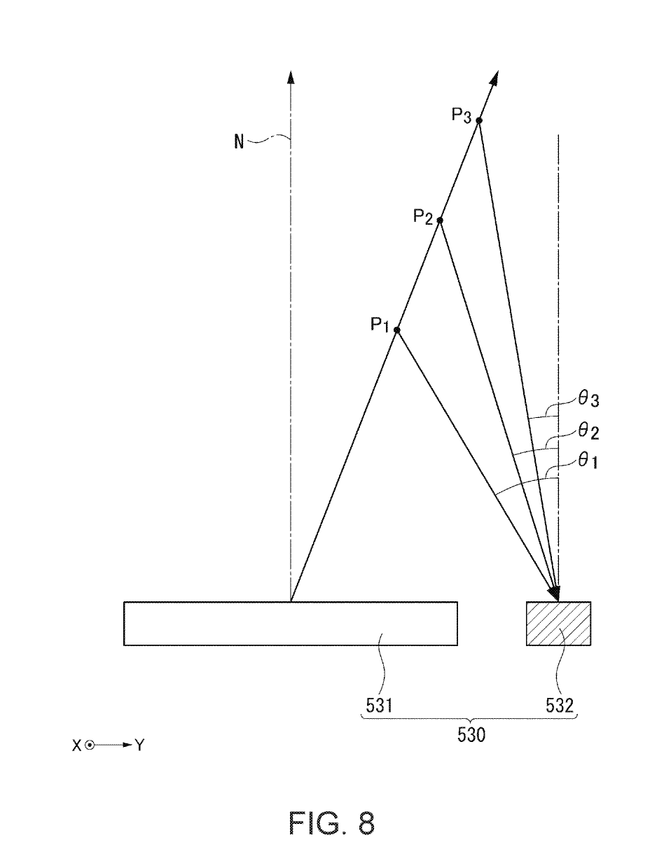

FIG. 7 is a diagram schematically showing an action of driving the ultrasonic element group 5 to transmit and receive the ultrasonic wave. FIG. 8 is a diagram schematically showing a comparative example in which the transmitting aperture and the receiving aperture are configured so as to be arranged side by side in the slicing direction. It should be noted that although the acoustic lens 8 is omitted in FIG. 7, the ultrasonic wave is converged in the slicing direction by the acoustic lens 8 in the present embodiment.

As shown in FIG. 7, when driving the transmitting elements 51 of the ultrasonic element group 5 at the same time to transmit the ultrasonic wave, the ultrasonic wave is converged by the acoustic lens 8. In the ultrasonic element group 5 of the present embodiment, the central position of the composite transmitting aperture 513 formed of the transmitting elements 51 and the central position of the receiving aperture 521 formed of the receiving elements 52 coincide with each other. Therefore, it is possible for the receiving elements 52 to receive the reflected wave from an object X located on or around the normal line N of the ultrasonic array passing through the central position. Thus, it is possible to inhibit the incident angle of the reflected wave to the receiving elements 52 from varying even in the case in which the position in the depth direction of the object is different.

In contrast, as shown in FIG. 8, in an ultrasonic element group 530 configured so that a transmitting aperture 531 and a receiving aperture 532 are arranged side by side in the slicing direction, when the generation position Pn of the reflected wave varies in the direction along the normal line N of an ultrasonic wave transmitting/receiving surface of the ultrasonic element group 530, namely the depth direction, the incident angle θn of the reflected wave to the receiving aperture 532 also varies (in FIG. 8, the three generation positions Pn (n=1, 2, 3) are illustrated). Therefore, it is necessary to calculate the position of the object taking the incident angle of the reflected wave into consideration in addition to the reception timing of the reflected wave, and there is a possibility that the process becomes complicated.

In contrast, in the ultrasonic element group 5 of the present embodiment shown in FIG. 7, since it is possible to inhibit the incident angle of the reflected wave to the receiving elements 52 from varying in accordance with the position in the depth direction of the object, it is possible to inhibit the process from becoming complicated due to the calculation of the position of the object with the variation of the incident angle taken into consideration.

### Configuration of Circuit Board

The circuit board 6 shown in FIG. 2 is provided with a drive signal terminal (not shown) and a common terminal (not shown), and the ultrasonic device 4 is connected to the circuit board 6 with the flexible board 7. Further, the circuit board 6 is connected to the control device 10 via the cable 3.

The circuit board 6 is provided with a driver circuit for driving the ultrasonic device 4, and so on. Specifically, as shown in FIG. 2, the circuit board 6 is provided with a selection circuit 61, a transmission circuit 62, and a reception circuit 63.

The selection circuit 61 selects the transmitting elements 51 to be connected to the transmission circuit 62 based on the control by the control device 10.

The transmission circuit 62 outputs a transmission signal, which represents the fact that the ultrasonic device 4 is made to transmit the ultrasonic wave via the selection circuit 61 due to the control by the control device 10. It should be noted that the transmitting elements 51 included in the ultrasonic element group 5 selected by the selection circuit 61 are driven in accordance with the output of the transmission signal, and transmit an ultrasonic wave.

The reception circuit 63 outputs a reception signal, which has been input from the ultrasonic sensor 22, to the control device 10. The reception circuit 63 is configured including, for example, a low-noise amplifier circuit, a voltage-controlled attenuator, a programmable-gain amplifier, a low-pass filter, an A/D converter, and a phasing addition circuit, and performs a variety types of signal processing such as conversion of the reception signal into a digital signal, elimination of a noise component, amplification to a desired signal level, and phasing addition processing on each of the channels, and then outputs the reception signal thus processed to the control device 10.

### Configuration of Control Device

As shown in FIG. 2, the control device 10 is configured including, for example, an operation section 11, a display section 12, a storage section 13, and a control section 14. As the control device 10, there can be used a terminal device such as a tablet terminal, a smartphone, or a personal computer, and the control device 10 can also be a dedicated terminal device for operating the ultrasonic probe 2.

The operation section 11 is a user interface (UI) for the user to operate the ultrasonic measurement apparatus 1, and can be formed of, for example, a touch panel or operation buttons disposed on the display section 12, a keyboard, or a mouse.

The display section 12 is formed of, for example, a liquid crystal display, and displays an image.

The storage section 13 stores a variety of programs and a variety of data for controlling the ultrasonic measurement apparatus 1.

The control section 14 is constituted by an arithmetic circuit such as a central processing unit (CPU), a processing circuit for performing each of the processes described later, and a storage circuit such as a memory. Further, the control section 14 reads and executes the variety of programs stored in the storage section 13 to thereby function as a transmission/reception control section 141, a harmonic processing section 142, and a signal processing section 143.

The transmission/reception control section 141 performs control of making the selection circuit 61 select the transmission channel T to be the driving object. Further, the transmission/reception control section 141 performs control of a generation and output process of the transmission signal on the transmission circuit 62. Further, the transmission/reception control section 141 performs control of frequency setting, gain setting, and so on of the reception signal on the reception circuit 63.

The harmonic processing section 142 extracts a harmonic component for each of the channels based on the reception signal of each of the channels.

The signal processing section 143 performs a variety of processes for obtaining a good tomographic image on the reception signal on which the harmonic process has been performed. As the variety of processes, there can be cited a nonlinear compression process such as a logarithmic conversion process for converting the expression format so that the maximum part and the minimum part of the signal strength of the reception signal are easily checked at the same time, a sensitive time control (STC) process for correcting the gain (luminance) in accordance with the propagation time (i.e., the depth) of the reflected wave, and so on. Further, the signal processing section 143 generates a variety of ultrasonic images such as a B-mode image or an M-mode image, and then makes the display section 12 display the ultrasonic images.

### Functions and Advantages of First Embodiment

The ultrasonic element groups 5 each have the transmitting elements 51 and the receiving elements 52, and are configured so as to be able to transmit and receive the ultrasonic wave, and are arranged along the X direction (the scanning direction), and function as a single transmission/reception channel. In each of the ultrasonic element groups 5, the centroid position of the receiving area in which the receiving elements 52 are disposed coincides with the centroid position (the central position P0) of the transmitting area in which the transmitting elements 51 are disposed, and the transmitting area and the receiving area overlap each other.

Here, in the case in which the transmitting area and the receiving area are arranged so as to be shifted from each other in the Y direction (the slicing direction) when viewed in the X direction, since it is necessary to transmit the ultrasonic wave so as to be tiled toward the receiving area in the slicing direction, there is a possibility that it is not achievable to sufficiently converge the transmission wave, and thus, the resolution degrades. In contrast, in the present embodiment, since the first area Ar1 and the second area Ar2 overlap each other, the degradation of the resolution described above can be suppressed.

Further, since the centroid position of the receiving area coincides with the centroid position of the transmitting area, the degradation of the resolution not only in the slicing direction but also in the scanning direction can more surely be suppressed.

Further, in the case in which the transmitting area and the receiving area are arranged so as to be shifted in the Y direction from each other when viewed in the X direction, the incident angle of the reflected wave varies in accordance with the distance from the receiving area to the reflection position in the direction along the normal line N as shown in FIG. 8. Therefore, it is necessary to perform the signal processing and the subsequent image processing on the reception signal (e.g., a setting process of the delay time of the reflected wave, and a correction process of the signal strength to the propagation distance) taking the variation of the incident angle into consideration when processing the reception signal, and there is a possibility that the process becomes complicated. In contrast, in the present embodiment, since the incident angle variation described above can be suppressed, and thus, the complication of the process described above can be suppressed, the ultrasonic image can be formed with a simple process.

Further, in the present embodiment, the transmitting elements 51 and the receiving elements 52 are disposed at the positions axisymmetrical about the center line L0. Thus, the symmetrical property in the Y direction, namely the slicing direction, of the transmitting area and the receiving area can be improved. Therefore, it is possible to improve the resolution in the slicing direction, and thus, the reception accuracy can be improved.

Further, the transmitting elements 51 and the receiving elements 52 are disposed at the positions point-symmetrical about the central position P0 (the centroid position of the transmitting area and the receiving area). Therefore, according to this configuration, the planar symmetry of the transmitting area and the receiving area can be improved. Therefore, it is possible to converge the ultrasonic wave transmitted from the transmitting elements 51 to thereby improve the resolution in the case of receiving the reflected wave, which has been reflected by the object, with the receiving elements 52, and thus, the reception accuracy can be improved.

Since the ultrasonic elements 50 are arranged along the X direction and the Y direction in the ultrasonic element group 5, the planar symmetry in the arrangement of the ultrasonic elements 50 can further be improved. Therefore, it is possible to transmit the ultrasonic wave three-dimensionally more uniform from the transmitting elements 51, and thus, the accuracy of the ultrasonic measurement can be improved.

### Modified Example of First Embodiment

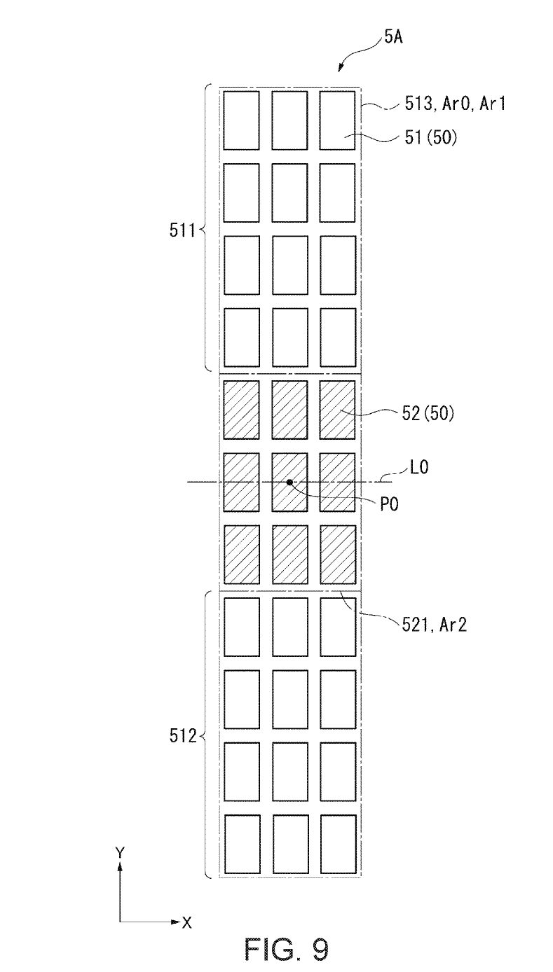

FIG. 9 is a plan view schematically showing an ultrasonic element group 5A related to a first modified example of the first embodiment.

Although in the first embodiment described above, the plurality of receiving elements 52 is arranged in a line along the X direction, in the ultrasonic element group 5A shown in FIG. 9, the plurality of receiving elements 52 is disposed so as to be arranged along the X direction and the Y direction. In FIG. 9, there is illustrated a configuration in which the 9 receiving elements 52 are disposed. In such a configuration, by increasing the number of receiving elements 52, the reception sensitivity can be improved.

### Second Embodiment

Then, a second embodiment according to the invention will be described.

In the first embodiment described above, the receiving elements 52 are disposed so as to be adjacent to each other. In contrast, the second embodiment is different in the point that the transmitting elements 51 are disposed on the X-direction side and the Y-direction side of the receiving element 52 in the ultrasonic element group.

It should be noted that in the following explanation, the constituents substantially the same as those of the first embodiment are denoted by the same reference symbols, and the explanation thereof will be omitted or simplified.

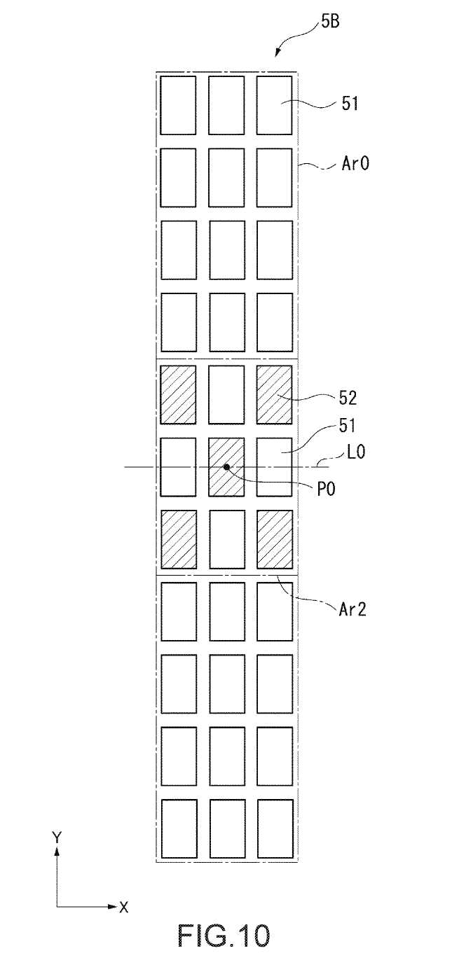

FIG. 10 is a plan view schematically showing an ultrasonic element group 5B of the second embodiment.

As shown in FIG. 10, regarding the plurality of receiving elements 52, the plurality of transmitting elements 51 and the plurality of receiving elements 52 are disposed in the second area Ar2 having a rectangular shape. The centroid position of the receiving area in which the receiving elements 52 are disposed coincides with the central position P0 of the transmitting/receiving area Ar0 of the ultrasonic element group 5B. Specifically, one of the receiving elements 52 is disposed at a position overlapping the central position P0. Further, the plurality of receiving elements 52 is disposed so as to be located on the both sides of each of the transmitting elements 51 in the X direction and the Y direction.

It should be noted that in the transmitting/receiving area Ar0, the centroid position of the transmitting area in which the transmitting elements 51 are disposed coincides with the central position P0 of the transmitting/receiving area Ar0 and the centroid position of the receiving area in which the receiving elements 52 are disposed. Further, the transmitting elements 51 and the receiving elements 52 are each disposed at the positions axisymmetrical about the center line L0.

### Functions and Advantages of Second Embodiment

In the second embodiment, since the transmitting elements 51 and the receiving elements 52 are alternately arranged in the X direction and the Y direction in the second area Ar2 set in the vicinity of the center of the transmitting/receiving area Ar0, the degradation of the output of the transmission wave from the vicinity of the central position P0 can be suppressed. Further, it is possible to suppress degradation of the image quality of the ultrasonic image due to the degradation of the planar symmetry in the arrangement of the receiving elements 52.

### Third Embodiment

Then, a third embodiment according to the invention will be described.

In the second embodiment, the transmitting elements 51 and the receiving elements 52 are arranged alternately in the X direction and the Y direction in the second area Ar2. In contrast, the third embodiment is different in the point that the receiving elements 52 are disposed along a diagonal line of the receiving area.

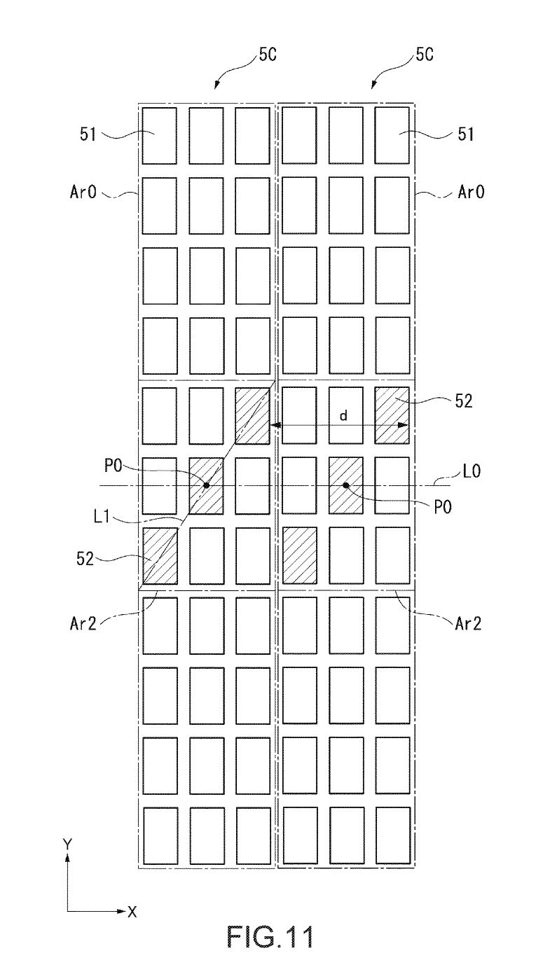

FIG. 11 is a plan view schematically showing an ultrasonic element group 5C of the third embodiment.

As shown in FIG. 11, the receiving elements 52 are arranged along the diagonal line L1 of the second area Ar2 so as to be shifted one at a time to the +X side and the +Y side. Further, one of the receiving elements 52 is disposed at a position overlapping the central position P0. The centroid position of the receiving area in which the receiving elements 52 are disposed coincides with the central position P0 of the transmitting/receiving area Ar0 of the ultrasonic element group 5C, and the receiving elements 52 are disposed at the positions point-symmetrical about the central position P0.

It should be noted that the centroid position of the transmitting area in which the transmitting elements 51 are disposed coincides with the central position P0, and the transmitting elements 51 are disposed at the positions point-symmetrical about the central position P0.

Further, the receiving elements 52 are arranged in the scanning direction at predetermined intervals d. In other words, the reception channels each included in one ultrasonic element group 5C are arranged in the scanning direction at the predetermined intervals d. It should be noted that the predetermined intervals d are set to the value corresponding to the frequency (the wavelength) of the high-order harmonic wave as the reception object with respect to the fundamental wave transmitted from the transmitting elements 51.

It should be noted that in the present embodiment, the configuration in which the receiving elements 52 are arranged so as to be shafted one at a time to the +X side and the +Y side is not a limitation, but a variety of types of arrangement configurations of the receiving elements 52 such as a configuration in which the receiving elements 52 are arranged so as to be shifted one at a time to the +X side and the −Y side can be adopted providing the receiving elements 52 are disposed point-symmetrically about the central position P0, and the receiving elements 52 are arranged at the predetermined intervals d in the scanning direction in the second area Ar2 in that configuration.

### Functions and Advantages of Third Embodiment

In the present embodiment, since the receiving elements 52 are arranged along the diagonal line L1 of the second area Ar2, it is possible to inhibit the output of the transmission wave from the vicinity of the central position P0 from degrading.

Further, the transmitting elements 51 and the receiving elements 52 are disposed at the positions point-symmetrical about the central position P0. Thus, it is possible to improve the planar symmetry related to the arrangement of the transmitting elements 51 and the receiving elements 52 in the transmitting/receiving area Ar0, and the receiving accuracy can be improved.

Further, the reception channels each configured by arranging the receiving elements 52 along the diagonal line L1 of the second area Ar2 are arranged in the scanning direction (the X direction) at the predetermined intervals d. By setting the predetermined intervals d in accordance with the frequency (wavelength) of the high-order harmonic wave as the reception object and so on, the reception accuracy of the high-order harmonic wave can be improved.

### Fourth Embodiment

Then, a fourth embodiment according to the invention will be described.

In each of the embodiments described above, the ultrasonic element group is provided with the ultrasonic elements of the same type capable of receiving a high-order harmonic wave of the predetermined order as the receiving elements 52. In contrast, the fourth embodiment is different from each of the embodiments in the point that there are provided a plurality of types of receiving elements configured so as to be able to appropriately receive the high-order harmonic waves different in order.

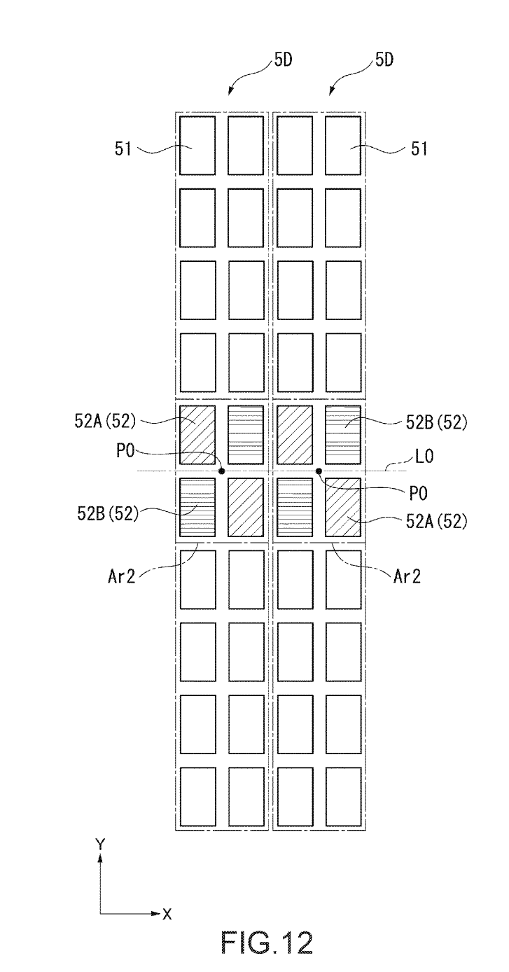

FIG. 12 is a plan view schematically showing an ultrasonic element group 5D of the fourth embodiment.

The ultrasonic element group 5D shown in FIG. 12 is provided with first receiving elements 52A and second receiving elements 52B configured so as to be able to receive the high-order harmonic waves of the respective orders different from each other as the receiving elements 52.

The first receiving elements 52A are the ultrasonic elements for receiving a high-order harmonic wave of a predetermined order, for example, a second-order harmonic wave with respect to the fundamental wave. Specifically, the first receiving elements 52A each have a shape (i.e., an aperture shape of the aperture part 411A) of the vibrating area of the vibrating film 412 and the characteristics of the piezoelectric elements 413 corresponding to the frequency (wavelength) of the second-order harmonic wave, and are each configured so as to be able to receive the second-order harmonic wave in good condition.

The second receiving elements 52B are the ultrasonic elements for receiving a high-order harmonic wave of an order different from that of the first receiving elements 52A, for example, a third-order harmonic wave, and are each configured so as to be able to receive the third-order harmonic wave in good condition.

In FIG. 12, the four receiving elements 52 including the first receiving elements 52A and the second receiving elements 52B constitute the second area Ar2. The centroid position of the receiving area in which the receiving elements 52 are disposed coincides with the central position P0 of the transmitting/receiving area Ar0 and the central position of the second area Ar2. In the second area Ar2, the first receiving elements 52A and the second receiving elements 52B are arranged alternately in the X direction and the Y direction. In such a manner, in the second area Ar2, the first receiving elements 52A and the second receiving elements 52B are each disposed at the positions point-symmetrical about the centroid position of the receiving area.

Further, the plurality of ultrasonic element groups 5D is arranged in the X direction, and the first receiving elements 52A and the second receiving elements 52B are arranged alternately along the X direction throughout the plurality of ultrasonic element groups 5D.

### Functions and Advantages of Fourth Embodiment

In the present embodiment, as the receiving elements 52, the first receiving elements 52A and the second receiving elements 52B for receiving the high-order harmonic waves of the respective orders different from each other are provided. Thus, it is possible to improve the reception accuracy with respect to the high-order harmonic waves of the respective orders different from each other.

Further, the first receiving elements 52A and the second receiving elements 52B are disposed at the positions point-symmetrical about the central position of the second area Ar2, namely the central position P0 in the present embodiment. Thus, it is possible to improve the planar symmetry of the arrangement positions with respect to each of the first receiving elements 52A and the second receiving elements 52B, and the high-order harmonic waves of the corresponding orders can be received with high accuracy.

### Modified Examples

It should be noted that the invention is not limited to each of the embodiments described above, but includes modifications and improvements within a range where the advantages of the invention can be achieved, and configurations, which can be obtained by, for example, arbitrarily combining the embodiments.

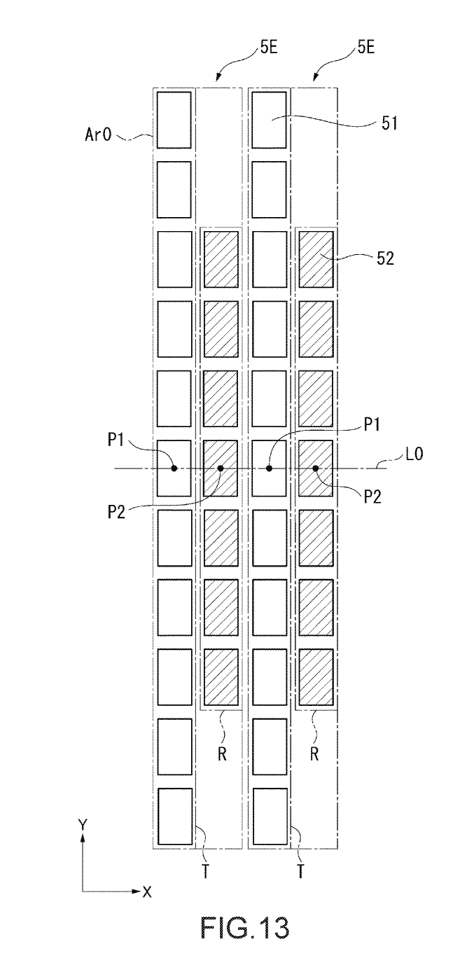

FIG. 13 is a diagram showing a schematic configuration of an ultrasonic element group in a modified example of the ultrasonic device according to the invention.

Although in each of the embodiments described above, there is illustrated the configuration of being provided with the transmitting elements 51 and the receiving elements 52 as the plurality of ultrasonic elements 50 arranged in the Y direction (the slicing direction), the invention is not limited to this configuration. Specifically, it is possible to adopt ultrasonic element groups 5E each constituted by a transmission channel T having the plurality of transmitting elements 51 arranged in the Y direction, and a reception channel R having the plurality of receiving elements 52 arranged in the Y direction arranged side by side in the X direction as shown in FIG. 13.

As shown in FIG. 13, the central position P1 of the transmission channel T is located on the center line L0 passing through the center of the transmitting/receiving area Ar0 having a rectangular shape surrounding the transmission channel T and the reception channel R. Similarly, the central position P2 of the reception channel R is located on the center line L0. In other words, in the projection view in the X direction, the centroid position of the transmitting area and the centroid position of the receiving area overlap each other. Further, in each of the ultrasonic element groups 5E, the transmitting elements 51 and the receiving elements 52 are disposed at the positions axisymmetrical about the center line L0.

Further, in the ultrasonic array constituted by the plurality of ultrasonic element groups 5E arranged in the Y direction, the transmission channels T and the reception channels R are each arranged in the Y direction at predetermined intervals. It should be noted that the predetermined intervals are intervals corresponding to the frequency of the high-order harmonic wave received by the reception channels R. In other words, the reception channels R are arranged at intervals equal to or smaller than the maximum interval between the reception channels R with which the high-order harmonic wave can be received with a desired accuracy.

In such a configuration, the respective central positions (the centroid positions) P1, P2 of the transmission channel T and the reception channel R are located on the center line L0 passing through the central position P1 of the transmitting/receiving area Ar0, and coincide with each other in the projection view in the scanning direction. Therefore, compared to the configuration (see FIG. 8) in which the transmission channels T and the reception channels R are arranged side by side in the slicing direction, the resolution in the slicing direction can be improved, and at the same time, the complication of the process related to the generation of the ultrasonic image based on the reception signal can be suppressed.

Further, by arranging the transmission channels T and the reception channels R side by side in the scanning direction, it is possible to increase the area of the receiving aperture while suppressing the reduction of the area of the transmitting aperture. Therefore, it is possible to improve the reception accuracy of the reflected wave while suppressing the degradation of the resolution, and the degradation of the image quality of the ultrasonic image can be suppressed.

Although in each of the embodiments described above, there is illustrated the configuration in which the centroid position of the receiving area coincides with the centroid position of the transmitting area in the Y direction (the slicing direction), the invention is not limited to this configuration. For example, it is possible to adopt a configuration in which the centroid position of the receiving area overlaps the transmitting area in the projection view in the X direction (the scanning direction). In such a configuration, compared to the case in which the transmitting area and the receiving area are shifted in the Y direction (the slicing direction) from each other when viewed in the X direction, the distance between the centroid position of the receiving area and the centroid position of the transmitting area, namely the central position of the first area Ar1 and the central position of the second area Ar2, can be shortened, and thus, the degradation of the resolution can be suppressed.

Although in each of the embodiments described above, in the projection view along the X direction (the slicing direction), there is illustrated the configuration in which the receiving area (the receiving aperture) is located inside the transmitting area (the composite transmitting aperture), the invention is not limited to this configuration. It is also possible that, for example, the receiving area and the transmitting area are shifted in the Y direction from each other within the range in which the centroid position of the receiving area overlaps the transmitting area, and a part of the receiving area is located outside the transmitting area in the projection view. In such a configuration, compared to the case in which the transmitting area and the receiving area are shifted in the Y direction (the slicing direction) from each other when viewed in the X direction, the distance between the centroid position of the receiving area and the centroid position of the transmitting area, namely the central position of the first area Ar1 and the central position of the second area Ar2, can be shortened, and thus, the degradation of the resolution can be suppressed.

Although in the fourth embodiment, there is illustrated the configuration provided with the two types of receiving elements 52, it is also possible to adopt a configuration simultaneously provided with three or more types of receiving elements 52 for receiving the high-order harmonic waves of the respective orders different from each other in good condition.

Further, although there is illustrated the configuration in which the two lines of receiving elements 52 each arranged along the X direction are arranged in the Y direction, it is also possible to adopt a configuration including only one line or a configuration having three or more lines arranged. Further, it is also possible to adopt a configuration in which the plurality of receiving elements 52 are arranged only in the Y direction. Further, it is also possible for the receiving elements 52 different in type not to be arranged alternately, but to be arranged adjacent to each other in the X direction and the Y direction.

Although in each of the embodiments described above, as the ultrasonic device, there is illustrated the configuration configured so as to drive the transmitting elements 51 constituting the ultrasonic element group at the same time to have the one-dimensional array structure, the invention is not limited to this configuration. Specifically, it is possible to adopt a configuration in which at least a part of the transmitting elements 51 can be driven individually in one ultrasonic element group. For example, the transmitting elements 51 arranged side by side in the scanning direction can be configured so as to individually be driven while being delayed in the slicing direction.

Although in each of the embodiments described above, there is illustrated the ultrasonic measurement device taking a living body as the measurement object, the invention is not limited to this example. For example, the invention can be applied to an electronic apparatus taking a variety of types of structures as the measurement object, and for performing the detection of the defects and inspection of aging of the structure. Further, the invention can also be applied to an electronic apparatus taking, for example, a semiconductor package or a wafer as the measurement object, and for detecting the defects of the measurement object.

Besides the above, specific structures to be adopted when implementing the invention can be configured by arbitrarily combining the embodiments and the modified examples described above with each other, or can arbitrarily be replaced with other structures and so on within the range in which the advantages of the invention can be achieved.

The entire disclosure of Japanese Patent Application No. 2015-171174 filed on Aug. 31, 2015 is expressly incorporated by reference herein.

What is claimed is:

1. An ultrasonic device comprising:
a plurality of element groups each including at least one transmitting element adapted to transmit an ultrasonic wave and at least one receiving element adapted to receive an ultrasonic wave, and arranged along a first direction,
wherein in each of the element groups, a centroid position of a receiving area, in which the receiving element included in the element group is disposed, overlaps a transmitting area, in which the transmitting element included in the element group is disposed, in a projection view along the first direction.

2. The ultrasonic device according to claim 1, wherein
the receiving area is located inside the transmitting area in the projection view along the first direction.

3. The ultrasonic device according to claim 1, wherein
the centroid position in the receiving area overlaps a centroid position of the transmitting area in the projection view along the first direction.

4. The ultrasonic device according to claim 3, wherein
the centroid position of the receiving area coincides with the centroid position of the transmitting area.

5. The ultrasonic device according to claim 4, wherein
the transmitting elements included in the element group are disposed at positions axisymmetrical about an imaginary line passing through the centroid position of the transmitting area and parallel to the first direction, and
the receiving elements included in the element group are disposed at positions axisymmetrical about the imaginary line.

6. The ultrasonic device according to claim 4, wherein
the transmitting elements included in the element group are disposed at positions point-symmetrical about the centroid position of the transmitting area, and
the receiving elements included in the element group are disposed at positions point-symmetrical about the centroid position of the transmitting area.

7. The ultrasonic device according to claim 6, wherein
the element group has an array structure in which ultrasonic elements including the transmitting element and the receiving element are arranged in a two-dimensional array along the first direction and a second direction crossing the first direction.

8. The ultrasonic device according to claim 1, wherein
the element groups each have a plurality of receiving elements,
the plurality of receiving elements includes at least one first receiving element adapted to receive a high-order harmonic wave taking the ultrasonic wave transmitted from the transmitting element as a fundamental wave, and at least one second receiving element adapted to receive a high-order harmonic wave of a different order from an order of the high-order harmonic wave received by the first receiving element,
the first receiving elements are disposed at positions point-symmetrical about the centroid position of the receiving area, and
the second receiving elements are disposed at positions point-symmetrical about the centroid position of the receiving area.

9. An ultrasonic module comprising:
an ultrasonic device including a plurality of element groups each including at least one transmitting element adapted to transmit an ultrasonic wave and at least one receiving element adapted to receive an ultrasonic wave, and arranged along a first direction; and
a circuit board on which the ultrasonic device is disposed,
wherein in each of the element groups, a centroid position of a receiving area, in which the receiving element included in the element group is disposed, overlaps a transmitting area, in which the transmitting element included in the element group is disposed, in a projection view along the first direction.

10. An ultrasonic measurement apparatus comprising:
an ultrasonic device including a plurality of element groups each including at least one transmitting element adapted to transmit an ultrasonic wave and at least one receiving element adapted to receive an ultrasonic wave, and arranged along a first direction; and
a control section adapted to control the ultrasonic device,
wherein in each of the element groups, a centroid position of a receiving area, in which the receiving element included in the element group is disposed, overlaps a transmitting area, in which the transmitting element included in the element group is disposed, in a projection view along the first direction.
